(19) United States
(12) Patent Application Publication
(10) Pub. No.: US 2021/0177459 A1
(43) Pub. Date: Jun. 17, 2021

(54) SEAL SYSTEM FOR AN ARTHROSCOPIC CANNULA

(71) Applicant: Conmed Corporation, Utica, NY (US)

(72) Inventors: Ryo Isshiki, Largo, FL (US); Jennifer Hicks, Cocoa, FL (US); Scott Padilla, Tampa, FL (US)

(73) Assignee: Conmed Corporation, Utica, NY (US)

(21) Appl. No.: 17/123,775

(22) Filed: Dec. 16, 2020

Related U.S. Application Data
(60) Provisional application No. 62/948,373, filed on Dec. 16, 2019.

(51) Int. Cl. A61B 17/34

(52) U.S. Cl. CPC A61B 17/3462; A61B 2017/00862

(57) ABSTRACT

A seal shield system protects and preserves seal functionality within a cannula during usage while maintaining existing fluid management and reduced drag. The seal shield system includes an end cap with an aperture extending therethrough, a cannula body extending distally from the end cap, a seal shield connected to the end cap, the seal shield having a first thickness, and one or more seals connected to the seal shield such that the seal shield is connected between the end cap and the one or more seals, one of the one or more seals having a second thickness. The first thickness is smaller than the second thickness.

### CROSS-REFERENCE TO RELATED APPLICATION

The present application relates and claims priority to U.S. Provisional Application No. 62/948,373 filed Dec. 16, 2019, the entirety of which is hereby incorporated by reference.

### BACKGROUND OF THE INVENTION

### 1. Field of the Invention

The present invention relates to a cannula and, more particularly, to a seal system for an arthroscopic cannula.

### 2. Description of Related Art

Cannulae may be used to support arthroscopic procedures by providing access and maintaining portals to a surgical site. To address issues of fluid management, cannulae may be equipped with a seal system at the proximal end. This seal produces an airtight seal around instrumentation with a circular profile and minimize aspiration of air into the working space when negative pressure is developed. Additionally, the seal system is designed to accommodate instrument and implant passage while controlling fluid loss.

Current seal systems have been found to be susceptible to damage when used with sharp and large instruments. Large instruments with sharp edges have been shown to significantly damage the seals when passed through the cannula. This can be observed in FIGS. 1A and 1B, which capture seals before (FIG. 1A) and after (FIG. 1B) use with a sharp instrument.

Therefore, there exists a need for a cannula system with a protective device for limiting damage to the seals.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### BRIEF SUMMARY OF THE INVENTION

Embodiments of the present invention are directed to a seal shield system for an arthroscopic cannula for limiting damage to seals, while preserving seal functionality. An embodiment of the seal shield system includes an end cap with an aperture extending therethrough, a seal shield connected to the end cap, the seal shield having a first thickness, and one or more seals connected to the seal shield such that the seal shield is connected between the end cap and the one or more seals, one of the one or more seals having a second thickness. The first thickness is smaller than the second thickness.

According to another aspect, the seal shield system includes an end cap with an aperture extending therethrough, a cannula body extending distally from the end cap, a seal shield connected to the end cap, the seal shield having a first thickness, and one or more seals connected to the seal shield such that the seal shield is connected between the end cap and the one or more seals, one of the one or more seals having a second thickness. The first thickness is smaller than the second thickness.

These and other aspects of the invention will be apparent from and elucidated with reference to the embodiment(s) described hereinafter.

### BRIEF DESCRIPTION OF THE SEVERAL VIEWS OF THE DRAWING(S)

The present invention will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1A is a top view of a seal of the prior art;

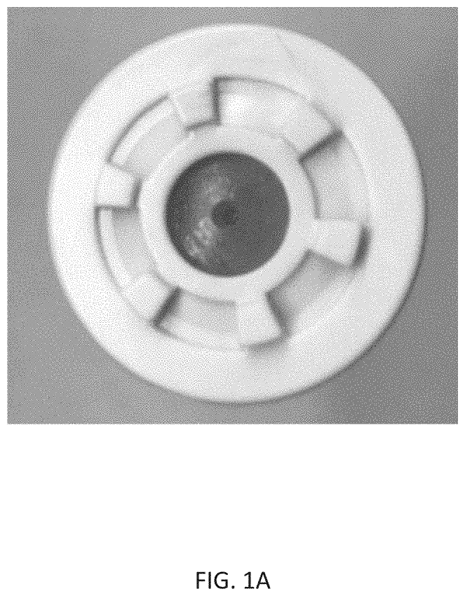

FIG. 1B is a top view of a damaged seal of the prior art;

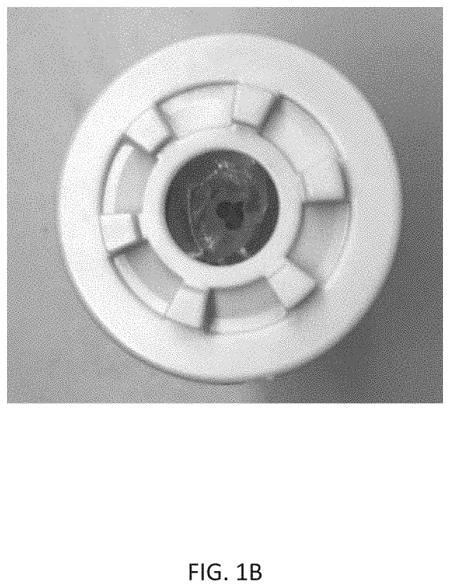

FIG. 2 is an exploded perspective view of a seal shield system, according to an embodiment; and

FIG. 3 is a side, sectional view of the proximal end of the seal shield system, according to an embodiment.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

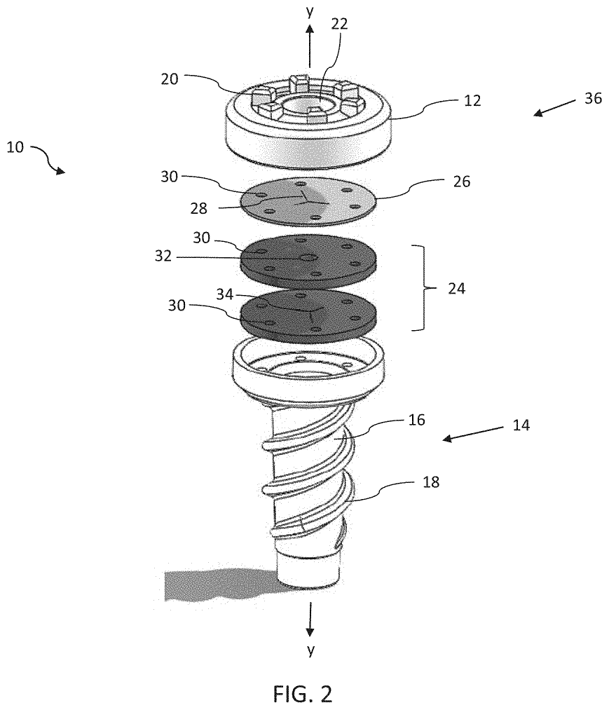

Referring now to the figures, wherein like reference numerals refer to like parts throughout, FIG. 2 is an exploded perspective view of a seal shield system 10, according to an embodiment. The seal shield system 10 includes a proximal end cap 12 with a cannula body 14 extending therefrom. The cannula body 14 is a tube 16 with an inner volume (not shown) extending therethrough. As shown in FIG. 2, the cannula body 14 has threads 18 extending along at least a portion of the tube 16.

The end cap 12 in FIG. 2 has a plurality of radial protrusions 20 extending proximally therefrom. The protrusions 20 are positioned radially around a central aperture 22 through the end cap 12. In the depicted embodiment, the central aperture 22 has a circular cross-section. The seal shield system 10 further comprises one or more seals 24 positioned between the end cap 12 and the cannula body 14. A seal shield 26 is positioned between the one or more seals 24 and the end cap 12.

In the depicted embodiment, the seal shield 26 is thin and durable. According to an embodiment, the seal shield 26 is composed of an elastomeric material with strong abrasion resistance properties. These characteristics of the seal shield 26 allow the seal shield 26 to protect the distal one or more seals 24 from large and sharp instruments. The seal shield 26 is compatible with any configurations of the one or more seals 24 that allows for the seal shield 26 to expand inside the distal one or more seals 24 and maintain a protective layer between the instrument seals (one or more seals 24) prone to damage.

Still referring to FIG. 2, the seal shield 26 comprises one or more slits 28 extending therethrough. In FIG. 2, the seal shield 26 has three converging slits 28 forming a Y-shape. The converging slits 28 are centrally located on the seal shield 26 such that the converging slits 28 are substantially aligned with the central aperture 22 through the end cap 12. Furthermore, the seal shield 26 has a plurality of radial apertures 30 extending therethrough around the converging slits 28. The radial apertures 30 allow for interconnection of the end cap 12, seal shield 26, and one or more seals 24 within the cannula body 14. According to an embodiment, each of the one or more seals 24 also have the plurality of radial apertures 30. Each of the one or more seals 24 has either a central aperture 32 or central converging slits 34 (as described above with reference to the seal shield 26). As shown, the central aperture 32 and converging slits 34 are encircled by the plurality of radial apertures 30. In FIG. 2, the central aperture 22 of the end cap 12, the converging slits 28 of the seal shield 26, and either the central aperture 32 or central converging slits 34 of the one or more seals 24 are substantially aligned along a central longitudinal y-y axis.

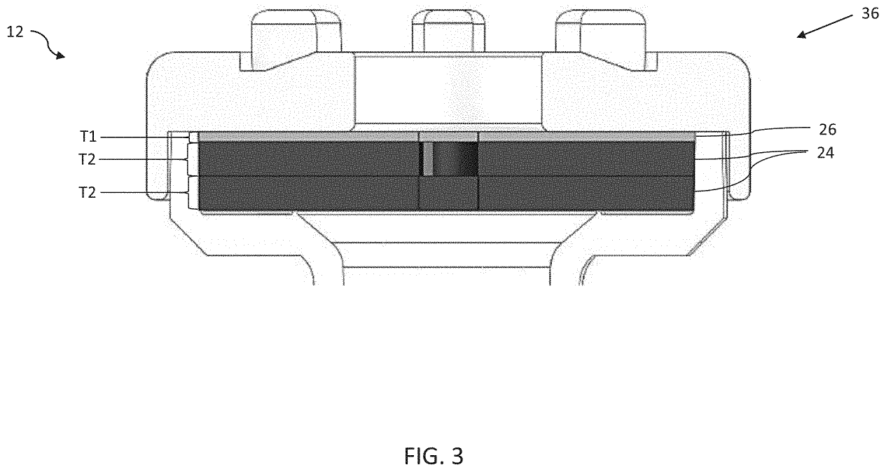

Turning now to FIG. 3, there is shown a side, sectional view of the proximal end 36 of the seal shield system 10, according to an embodiment. As shown in FIG. 3, the seal shield 26 has a first thickness T1 and each of the one or more seals 24 has a second thickness T2. In the depicted embodiment, T2 is greater than T1. In other words, the seal shield 26 is thinner than each of the one or more seals 24. In alternative embodiment, the seal shield 26 is thinner than at least one of the one or more seals 24 (i.e., seal shield 26 is thinner than one of the one or more seals 24 but not thinner than another of the one or more seals 24). The relative thickness (T1<T2) of the seal shield 26 and the one or more seals 24 maintains the function of the cannula body 14. Furthermore, the thin seal shield 26 does not add additional drag force and allows for the intended function to be maintained.

It should be understood that the values used above are only representative values, and other values may be in keeping with the spirit and intention of this disclosure.

While several inventive embodiments have been described and illustrated herein with reference to certain exemplary embodiments, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein (and it will be understood by one skilled in the art that various changes in detail may be effected therein without departing from the spirit and scope of the invention as defined by claims that can be supported by the written description and drawings). More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Further, where exemplary embodiments are described with reference to a certain number of elements it will be understood that the exemplary embodiments can be practiced utilizing either less than or more than the certain number of elements.

All references, including publications, patent applications, and patents, cited herein are hereby incorporated by reference to the same extent as if each reference were individually and specifically indicated to be incorporated by reference and were set forth in its entirety herein.

All definitions, as defined and used herein, should be understood to control over dictionary definitions, definitions in documents incorporated by reference, and/or ordinary meanings of the defined terms.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if not directly attached to where there is something intervening.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

The recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed.

No language in the specification should be construed as indicating any non-claimed element as essential to the practice of the invention.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A seal shield system, comprising:
an end cap with an aperture extending therethrough;
a seal shield connected to the end cap, the seal shield having a first thickness;
one or more seals connected to the seal shield such that the seal shield is connected between the end cap and the one or more seals, one of the one or more seals having a second thickness; and
wherein the first thickness is smaller than the second thickness.

2. The system of claim 1, further comprising a plurality of protrusions extending proximally from the end cap around the aperture.

3. The system of claim 1, further comprising one or more slits extending through the seal shield.

4. The system of claim 3, wherein the one or more slits is three converging slits.

5. The system of claim 4, further comprising a plurality of radial apertures extending through the seal shield.

6. The system of claim 5, wherein the plurality of radial apertures are positioned around the three converging slits.

7. The system of claim 1, further comprising a central aperture extending through at least one of the one or more seals.

8. The system of claim 1, further comprising three converging slits extending through at least one of the one or more seals.

9. The system of claim 1, wherein the seal shield is composed of an elastomeric material.

10. A seal shield system, comprising:
an end cap with an aperture extending therethrough;
a cannula body extending distally from the end cap;
a seal shield connected to the end cap, the seal shield having a first thickness;
one or more seals connected to the seal shield such that the seal shield is connected between the end cap and the one or more seals, one of the one or more seals having a second thickness; and
wherein the first thickness is smaller than the second thickness.

11. The system of claim 10, wherein the cannula body comprises a tube with threads extending along at least a portion thereof.

12. The system of claim 10, further comprising a plurality of protrusions extending proximally from the end cap around the aperture.

13. The system of claim 10, further comprising one or more slits extending through the seal shield.

14. The system of claim 13, wherein the one or more slits is three converging slits.

15. The system of claim 14, further comprising a central aperture extending through at least one of the one or more seals or three converging slits extending through at least one of the one or more seals.

16. The system of claim 15, wherein the three converging slits in the seal shield are substantially aligned with the aperture of the end cap.

17. The system of claim 16, wherein the central aperture extending through at least one of the one or more seals or the three converging slits extending through at least one of the one or more seals is substantially aligned with the aperture of the end cap.

18. The system of claim 14, further comprising a plurality of radial apertures extending through the seal shield.

19. The system of claim 18, wherein the plurality of radial apertures are positioned around the three converging slits.

20. The system of claim 10, wherein the seal shield is composed of an elastomeric material.
